(19) United States
(12) Patent Application Publication
(10) Pub. No.: US 2026/0027492 A1
(43) Pub. Date: Jan. 29, 2026

(54) Hollow Fiber Membrane Module for Anti-Solvent Crystallization

(71) Applicant: New Jersey Institute of Technology, Newark, NJ (US)

(72) Inventors: Kamalesh Sirkar, Bridgewater, NJ (US); Yufeng Song, Newark, NJ (US); Lixin Feng, Kearny, NJ (US); Siddharth Tripathi, Hillsborough, NJ (US)

(73) Assignee: New Jersey Institute of Technology, Newark, NJ (US)

(21) Appl. No.: 19/278,283

(22) Filed: Jul. 23, 2025

Related U.S. Application Data
(60) Provisional application No. 63/674,367, filed on Jul. 23, 2024.

(51) Int. Cl. B01D 9/00; A61K 9/14; A61K 31/343; B01D 63/02; B01D 63/04; B01D 69/02; B01D 71/26

(52) U.S. Cl. CPC B01D 9/0054; A61K 9/145; A61K 31/343; B01D 63/02; B01D 63/046; B01D 69/02; B01D 71/262; B01D 2009/0086; B01D 2315/10; B01D 2325/36; B01D 2325/38

(57) ABSTRACT

A porous hollow fiber membrane based anti-solvent crystallization (AsCr) process is disclosed. The process involves injecting an anti-solvent from a bore of a hollow fiber membrane into a shell side where a feed solution containing a solution containing a material, e.g., an API, is flowing. The shell-side feed solution flows perpendicular to the hollow fiber length; the shell side liquid is in cross flow across the hollow fiber membranes. Multiple HFM modules may be positioned in series with nanocrystal suspension product from one module fed to the next module that is independently fed with an anti-solvent. The crossflow HFM based continuous AsCr technique disclosed herein could result in continuous nanocrystal production of APIs.

### CROSS-REFERENCE TO RELATED APPLICATION

The present application claims priority benefit to a US provisional patent application entitled “Hollow Fiber Membrane Module for Anti-Solvent Crystallization,” which was filed on Jul. 23, 2024, and assigned Ser. No. 63/674,367. The entire content of the foregoing US provisional patent application is incorporated herein by reference.

### STATEMENT REGARDING FEDERALLY SPONSORED RESEARCH

This invention was made with government support under Agreement No. 1822130 awarded by the National Science Foundation. The government has certain rights in the invention.

### FIELD OF THE DISCLOSURE

The present disclosure relates to a porous hollow fiber membrane-based continuous cross-flow anti-solvent crystallizer to produce active pharmaceutical ingredient (API) nanocrystals.

### BACKGROUND

Organic synthesis-based pharmaceuticals are generally produced in large scale by batch processes. Batch processing of APIs suffers from many shortcomings, such as large batch reactors and separators requiring high capital investments. Continuous manufacturing of APIs is an alternative to batch processing. Continuous manufacturing may employ small devices at one site that are connected serially, with quality of intermediates monitored at every step to enhance product quality, increase yield, and drastically reduce production time. Regardless of batch or continuous manufacturing, API crystallization is usually the final purification step.

In pharmaceutical crystallization, continuous manufacturing may be implemented by adopting mixed suspension mixed product removal (MSMPR) in a single stage or serially-connected multiple stages. [See, Chen, J., et al., Pharmaceutical crystallization, Cryst. Growth Des., 11, 887-895 (2011)] In addition, for anti-solvent crystallization (AsCr), a plug flow reactor (PFR) format with 3 to 4 distinct anti-solvent injection locations may be implemented. [See, Alvarez, A. J., et al., Continuous plug flow crystallization of pharmaceutical compounds, Cryst. Growth Des., 10 (5), 2219-2228 (2010)]. The crystal size distribution (CSD) range observed in the Alvarez publication was 10-100 μm+. The CSD could be increased/decreased with increased antisolvent addition points, depending on system kinetics.

Crystallization in MSMPR stirred vessels produces a broad crystal size distribution. A technique developed in response to this phenomenon is impinging jet crystallization [See, U.S. Pat. No. 5,314,506A to Midler et al., 1994], which results in a narrow crystal size distribution. This method has limitations due to jet alignment issues, limitation of the viscosity ratio of the two fluids, and scale-up.

A porous hollow fiber membrane-based method has been developed to overcome many such limitations. [See, Zarkadas, D. M., Crystallization Studies in Hollow Fiber Devices, PhD Thesis, New Jersey Institute of Technology, New Jersey, 2004; Zarkadas, D. M., et al., Anti-solvent crystallization in porous hollow fiber devices, Chem. Eng. Sci. 61, 5030-5048 (2006)]. In such membrane-based techniques, the solvent containing the solute flows on one side of a porous hollow fiber membrane parallel to the length of the hollow fiber membrane in laminar flow, and the anti-solvent-which flows on the other side of the hollow fiber membrane under laminar flow conditions—is injected through numerous membrane pores into the solvent containing the solute to be crystallized. The high level of mixing achieved in this design leads to a mixing scale close to the Kolmogorov scale (10-50 μm) for low viscosity liquids. Little energy input is necessary compared to that in conventional devices. The high mixing efficiency achieved in this configuration was modeled using Computational Fluid Dynamics (CFD) for water as anti-solvent coming through hollow fiber membrane pores into acetone flowing parallel to the hollow fiber membranes on the shell side. [See, Chen, D., et al., Hydrodynamic modeling of porous hollow fiber anti-solvent crystallizer for continuous production of drug crystals, J. Membrane Sci., 556, 185-195 (2018)].

Griseofulvin crystallization in the size range of 1.6 to 11.8 μm from an acetone solution in parallel flow along hollow fiber membranes has been achieved as anti-solvent water flowing in the hollow fiber membrane bore was injected into the shell side [See, Chen, D., et al., Continuous synthesis of polymer-coated drug particles by a porous hollow fiber membrane-based antisolvent crystallization, Langmuir 31, 432-441 (2015)]. The same configuration has been used for indomethacin crystallization from an alcohol solution flowing parallel to the hollow fiber membranes by anti-solvent water injected from a hollow fiber membrane bore [See, Fern, J. C. W., et al., Continuous synthesis of nano-drug particles by antisolvent crystallization using a porous hollow-fiber membrane module, International Journal of Pharmaceutics, 543, 139-150 (2018)].

In anti-solvent crystallization, the mean crystal size and the crystal size distribution—the two quantities of primary importance in API crystallization—are controlled by mixing efficiency, supersaturation, and residence time. Literature review suggests: (1) crystal size increases with residence time allowing crystals to grow; (2) increased supersaturation yields higher nucleation rates and a crystal size distribution of smaller mean size; and (3) efficient mixing is essential to achieve a smaller mean size and a narrower crystal size distribution. Therefore, efficient mixing and a low residence time under high supersaturation can lead to smaller mean crystal size and a lower crystal size distribution.

Nanocrystals are of increasing interest from drug delivery and bioavailability concerns. Among a number of important physicochemical properties of APIs, drug solubility and drug permeability through cell membranes are of primary importance. In fact, these two properties as described by the biopharmaceutical classification system (BCS) are at the heart of many efforts in drug dosage form development. Drugs, which are members of BCS classes II and IV, have poor water solubility resulting in poor bioavailability. A very large majority of drugs have poor aqueous solubility; hence, smaller crystal sizes have been a goal, since crystal surface area for dissolution is increased via nanonization.

An effective strategy for such nanocrystal development, which does not rely on reduction in solute concentration in hollow fiber membrane-based systems, is needed. More generally, there remains a need for a continuous crystallization process that can efficiently produce pharmaceutical nanocrystals with controlled size distribution, while overcoming the limitations of existing crystallization techniques.

### SUMMARY

In accordance with embodiments of the present disclosure, system(s) and method(s) for continuous anti-solvent crystallization of active pharmaceutical ingredients are provided. In one embodiment, the system includes a compact hollow fiber membrane module containing hollow fibers arranged perpendicular to an incoming shell-side flow of fluid containing the active pharmaceutical ingredient to be crystallized to create a cross-flow configuration.

In one embodiment, a process for continuous anti-solvent crystallization of active pharmaceutical ingredients is disclosed. The process includes providing a hollow fiber membrane module that includes a plurality of porous hollow fibers arranged in a cross-flow configuration relative to a shell-side fluid flow. An API solution is flowed through a shell-side of the hollow fiber membrane module. An anti-solvent is flowed through a tube side of the hollow fibers. The anti-solvent is permeated through pores of the hollow fibers into the shell-side, thereby mixing with the API solution. The mean flow length may be a predetermined length. In exemplary embodiments, the mean flow length is between 0.5 and 500 cm. The flow rates of the API solution and the anti-solvent are controlled to achieve a desired residence time of the API solution. Crystallization of the API is induced to form nanocrystals.

In exemplary embodiments of the disclosed porous hollow fiber membrane based anti-solvent crystallization system/technique, the anti-solvent is injected from the hollow fiber membrane bore into an active pharmaceutical ingredient-containing solution flowing in the shell side. In distinct contrast to conventional systems where the shell-side feed solution flows along the hollow fiber membrane, the disclosed system/method is configured such that the shell-side liquid flows perpendicular to the hollow fiber membrane. The mean flow length may be on the order of 3 cm, thereby reducing the residence time which strongly influences crystal growth.

In exemplary embodiments, anti-solvent crystallization of griseofulvin from its acetone solution has been undertaken using anti-solvent water injected through the hollow fiber membrane pores. The range of residence time may be on the order of 5-30 seconds. The volume flow rate ratio of acetone/water may be between 0.35 to 1.66. Griseofulvin concentration may be varied between saturation (3.73 g/100 g acetone) to 0.4 g GF in 100 g of solution having 50 wt % acetone in the total solution. Low residence time yielded nanocrystals <100 nm. The average API crystal size was about 230 nm in a three-hour operation. At a fixed mass volume flow rate ratio of 1.34±0.1, the average API crystal size increased linearly from 48 to 280 nm as residence time increased from 5 to 28 seconds.

For two porous hollow fiber membrane modules arranged in series with the nanocrystal suspension from one module fed to the next module that is independently fed with anti-solvent water, griseofulvin crystallization yields as high as 98% were achieved.

Anti-solvent crystallization of L-glutamic acid in water with anti-solvent acetone in a porous hollow fiber membrane module as disclosed herein further demonstrated continuous nanocrystal production and similar behaviors vis-à-vis variation of residence time and volume flow rate ratio.

The disclosed cross-flow hollow fiber membrane based continuous anti-solvent crystallization systems and methods are effective in continuously producing nanocrystals of APIs.

Any combination and/or permutation of the embodiments is envisioned. Other objects and features will become apparent from the following detailed description considered in conjunction with the accompanying drawings. It is to be understood, however, that the drawings are designed as an illustration only and not as a definition of the limits of the present disclosure.

### BRIEF DESCRIPTION OF THE DRAWINGS

To assist those of skill in the art in making and using the disclosed hollow fiber membrane system/method, reference is made to the accompanying figures, wherein:

FIG. 1A is a cut-away view of a hollow fiber membrane module (HFM) and operational configuration of the HFM-based anti-solvent device for crystallization with top end of the bore of the HFMs sealed by epoxy, in accordance with one embodiment of the present disclosure;

FIG. 1B shows the external dimensions of the exemplary HFM module of FIG. 1A (thickness is 1.27 cm (0.5 in));

FIG. 2A schematically depicts an exemplary system for continuous anti-solvent crystallization of griseofulvin;

FIG. 2B schematically depicts an exemplary system for continuous anti-solvent crystallization of L-glutamic acid nanocrystals;

FIG. 2C schematically depicts an exemplary system for continuous anti-solvent crystallization of an API that includes two HFM modules in series;

FIG. 3 is a plot showing saturation concentration of griseofulvin (GF) in acetone-water solution (% g/g of solution) vs. % weight ratio of acetone/total solvent (w/w) and total GF concentration in mixed acetone and antisolvent mixture in crystallizer for the experimental results of FIG. 7 (below);

FIG. 4 is a bar chart showing average GF crystal size and standard deviation from mean size after suspension in DI water/5 mM SDS solution (data in Table 2);

FIG. 5A is a plot showing post filtration crystal size diameter (CSD) of GF; flow rates (GF-acetone 9.6 g/min; water 7.4 g/min); estimated tᵣₑₛ, 8.5 s (2 cc vol);

FIG. 5B is a plot showing post filtration CSD of GF; flow rates (GF-acetone 4.28 g/min; water 3.75 g/min); estimated tᵣₑₛ, 18.7 s (2 cc vol);

FIG. 5C is a plot showing Post filtration CSD of GF; flow rates (GF-acetone 3.7 g/min; water 7.4 g/min); estimated tᵣₑₛ, 18.6 s (2 cc vol);

FIG. 6 is a plot showing residence time effect on GF crystal size in nm (in 5 mM SDS) for a fixed mass flow rate ratio (1.34±0.1) (the average crystal size is shown);

FIG. 7 is a plot showing average GF crystal size dependence on the mass flow rate ratio of acetone/water (w/w) for similar residence times; the residence time is identified next to the data point; all data were gathered from resuspension in 5 mM SDS solution;

FIG. 8 is a plot showing CSD of samples collected from a filtration cup in an experiment with feed sample 4 (50% by wt. of GF saturation concentration) flowing at 1 g/min and antisolvent water flowing in at 9.5 g/min for a mass flow rate ratio of (1/9.5); all data were gathered from resuspension in 5 mM SDS solution;

FIG. 9 is a bar chart showing average crystal size vs. particular time when a sample was taken and analyzed for an extended duration continuous crystallization run for 3 hr: GF-acetone flow rate, 3.35 mL/min; water flow rate 2.02 mL/min; volume FRR (acetone/water) of 1.66; mass FRR, 1.33; average residence time, 29.1 s; all data were gathered from resuspension in 5 mM SDS solution;

FIG. 10A is PXRD of GF nanocrystals;

FIG. 10B is Raman spectra of griseofulvin;

FIG. 10C is a set of scanning electron micrograph (SEM) images for GF crystallization filter and GF crystals on top of it;

FIG. 11 is a plot showing saturation concentration of L-GA in water-acetone solution (g/100 g of solution) vs. weight ratio of water/total solution (w/w);

FIG. 12 is a plot showing residence time effect on L-GA crystal size in nm for a fixed mass flow rate ratio (˜1.3); the average crystal size is shown for each data point;

FIG. 13 is a plot showing average L-GA crystal size dependence on the mass flow rate ratio of water/acetone (w/w) for similar residence times (numerical values marked in the figure for each data point);

FIG. 14 is a bar chart showing average crystal size of L-GA at various times during a run lasting 3 hours; the crystal collection time was 0.5 min; flow rate ratio was 0.98; residence time 30.6 s; and

FIG. 15 is a plot showing average crystal size of L-GA for 15 s residence time and various mass flow rate ratios, effect of collection time (5 s, 1 min) on filter in the filtration funnel.

### DETAILED DESCRIPTION

System(s) and method(s) for continuous anti-solvent crystallization of active pharmaceutical ingredients are provided. The disclosed system may include a compact hollow fiber membrane module containing hollow fibers arranged perpendicular to an incoming shell-side flow of fluid. The incoming fluid may contain an active pharmaceutical ingredient to be crystallized. The disclosed system is configured to create a cross-flow configuration.

The disclosed method may provide a process for continuous anti-solvent crystallization, e.g., for continuous crystallization of active pharmaceutical ingredients using an anti-solvent. The process may include providing a hollow fiber membrane module that includes a plurality of porous hollow fibers arranged in a cross-flow configuration relative to a shell-side fluid flow. An API solution may be flowed through a shell-side of the hollow fiber membrane module. An anti-solvent may be flowed through a tube side of the hollow fibers. In exemplary implementations of the disclosed process, the anti-solvent is permeated through pores of the hollow fibers into the shell-side, thereby mixing with the API solution.

The mean flow length of the system may be a predetermined length. In exemplary embodiments, the mean flow length may be between 0.5 and 500 cm. The flow rates of the solution, e.g., the API solution, and the anti-solvent may be controlled to achieve a desired residence time of the solution as well as creating a certain level of supersaturation. Crystallization of the API may be induced to form nanocrystals.

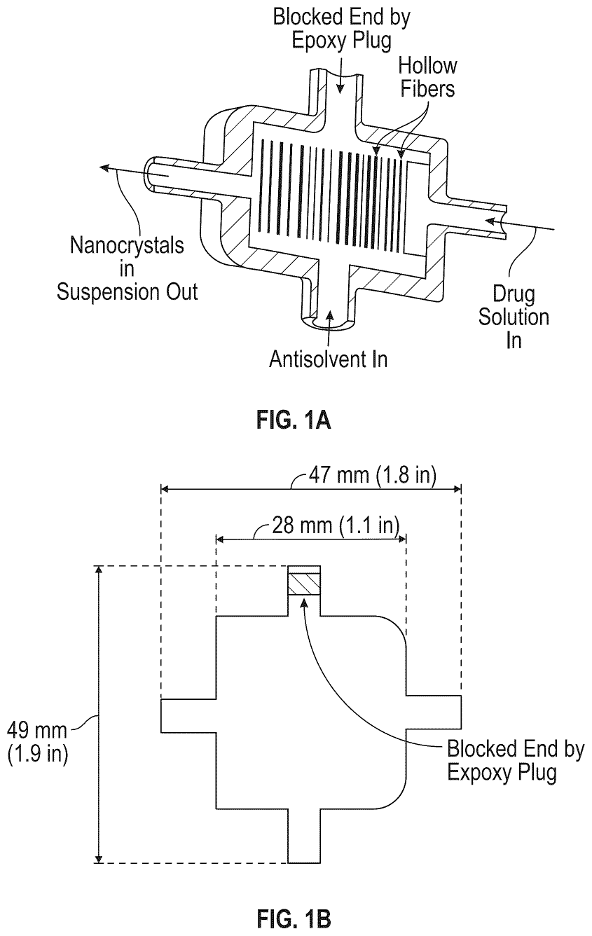

FIGS. 1A and 1B are schematic depictions of one embodiment of a crossflow based rectangular hollow fiber membrane device according to the present disclosure. FIG. 1A is a cut-away view of an exemplary HFM module and operational configuration of the HFM-based anti-solvent device for crystallization with top end of the bore of HFMs sealed by epoxy. FIG. 1B schematically depicts the external dimensions of such an exemplary HFM module. The thickness of the HFM module may be on the order of 1.27 cm (0.5 in).

The HFM module/device includes a housing with hollow fiber membranes, e.g., polypropylene hollow fiber membranes. It will be understood that other hollow fiber materials may be used; the fibers may be hydrophobic as in polypropylene, polyethylene, polytetrafluorethylene, polyvinylidene fluoride etc. or hydrophilic as in nylon, regenerated cellulose, polyvinyl alcohol etc. The housing includes a shell-side inlet to allow a solution, e.g., a drug solution, to enter and a shell-side outlet to allow the solution, e.g., the drug solution, to exit. The housing also includes/defines a tube-side inlet on a bottom end to allow the anti-solvent to enter, and a blocked top end.

The membrane device is sized to generate a very high level of mixing in the low Reynolds number flow of the shell-side API-containing liquid as the anti-solvent flowing in the hollow fiber membrane (HFM) bore is injected into it. The shell-side liquid flows across quite a few layers of hollow fiber membranes with vigorous mixing around each hollow fiber membrane resulting from intense mixing due to injection of the antisolvent and flow separations around each hollow fiber membrane. The shell-side liquid flows across a plurality of layers of the HFMs with vigorous mixing around each HFM resulting from anti-solvent injection into the API solution and flow separations around each HFM. Further, the shell-side device length, e.g., ˜3.175 cm (1.25 in) (e.g., total length including the outlets may be ˜4.57 cm (1.8 in)) allows achieving a short residence time. It will be understood that the shell-side device length and the total length can vary. Further, as noted above, the thickness of the module shown in FIG. 1B may be on the order of 0.5 in, but can be much larger. This device configuration may be scaled up to support greater flow volumes.

Crossflow around HFMs in membrane contactors have been shown to achieve shell-side liquid-phase mass transfer coefficients ten (10) times higher than that with parallel flow along HFMs in gas-liquid contactors that are small [see, S. R. Wickramasinghe, et al., Mass transfer in various hollow fiber geometries, J. Membrane Sci., 69 (1992) 235-250] or large [see, A. Sengupta, et al., Large-scale application of membrane contactors for gas transfer from or to ultrapure water, Separation and Purification Technology, 14 (1998) 189-200]. When this high level of mixing is imposed on the mixing generated by anti-solvent injection into the flowing solvent on the shell side, the results can be extraordinary.

In crystallization of griseofulvin from an acetone solution flowing on the shell side of a porous hollow fiber membrane module parallel to the hollow fiber membranes by anti-solvent water coming from the tube-side, the shell-side liquid residence time (reduced significantly by injection of anti-solvent into it) and the level of micromixing contribute to determination of the median size of the drug crystals. These two aspects will influence the nucleation rate and the growth rate in the crystallization process via the rate at which supersaturation is generated and consumed. To achieve a very small median crystal size and a low breadth of crystal size distribution, a high level of micromixing is necessary to generate high supersaturation leading to a high nucleation density and maintain a very low residence time to limit growth of already-formed nuclei. In distinct contrast to HFM systems/methods of the present disclosure, conventional hollow fiber membrane devices used for AsCr employ shell-side flow parallel to hollow fiber membrane length, which practically limits the reduction of HFM length and the residence time. The device of FIGS. 1A and 1B allows a short residence time for appropriate flow rates and ensures a high level of meso- and micro-mixing.

The materials and the methods of the present disclosure used in one embodiment will be described below. While the embodiment discusses the use of specific compounds and materials, it is to be understood that the present disclosure could employ other suitable compounds or materials. Similar quantities or measurements may be substituted without altering the method embodied below.

### Experimental Test/Results

The chemicals used in exemplary embodiments were:
acetone (Sigma Aldrich, PA);
Griseofulvin (Sigma Aldrich, PA);
L-glutamic acid (Fisher Scientific);
sodium dodecyl sulfate (Aldrich, PA);
ethanol (Acros Organics, 99.5% ACS reagent); and
deionized (DI) water.

For filtration of the crystallized solution, the following membranes were used:
Flat polyetheretherketone (PEEK) membrane filter (Novamem, Switzerland) of 0.02 μm pore size;
Al₂O₃ membrane of 0.02 μm size (Sterlitech, WA.; Whatman® Anodisc) for filtration of the crystallized solution for griseofulvin;
PEEK and Al₂O₃ membrane of 0.02 μm pore size for filtration of L-glutamic acid crystals.
Flat Nylon filter of 0.45 μm (#BLA045, 3M, Saint Paul, MN) to eliminate undissolved solids during the preparation of the saturated drug solution.

The hollow fiber membrane device schematically depicted in FIGS. 1A and 1B used for crystallization was a 0.5×1 Micromodule™ in high-density (HD) polyethylene (PE) housing (3M, Charlotte, NC; now Solventum, Charlotte, NC). The Micromodule membrane has 700 microporous hydrophobic X-50 hollow fiber membranes of polypropylene (PP) of length 3 cm perpendicular to the incoming shell-side flow of liquid containing the API to be crystallized. This device has the following hollow fiber membrane dimensions and properties:
0.022 cm (internal diameter (ID)),
0.030 cm (outer diameter (OD));
wall porosity, 0.4; pore size, 4×10⁻⁶ cm.
The shell-side priming volume located in a HD PE housing is ˜2 cm³ [See 3M website].

A cut-away view of the module is shown in the HD PE casing in FIG. 1A, in which the hollow fiber membranes are normally open at both ends from bottom to top; however, in this embodiment, the top end was sealed. The casing physical dimensions are shown in FIG. 1B.

FIG. 1A illustrates the operational configuration of an exemplary HFM module/device with the top end of the HFM tube side blocked with epoxy. It will be understood that other sealing mechanisms may be employed. The right-hand side of FIG. 1A shows drug solution flowing into the module in cross flow over the outside of the HFMs and exiting as a drug nanocrystal suspension on the left-hand side of FIG. 1A. The module bottom allows anti-solvent entry into the HFM bore. The blocked HFM bore exit at the top end forces the anti-solvent to come through HFM pores into the drug solution on the shell-side in cross flow, creating rapidly high supersaturation, and highly efficient mixing. Further, the shell-side residence time can be quite low since the shell-side solution is not flowing parallel to the HFM length.

The integrity of the hollow fiber membrane in the Micromodule™ was checked first for leakage by passing deionized water for a few hours through the tube side. This was done before blocking the exiting end of the hollow fiber membranes with epoxy. If there was no water leak from the shell side, the hollow fiber membranes were considered uncompromised.

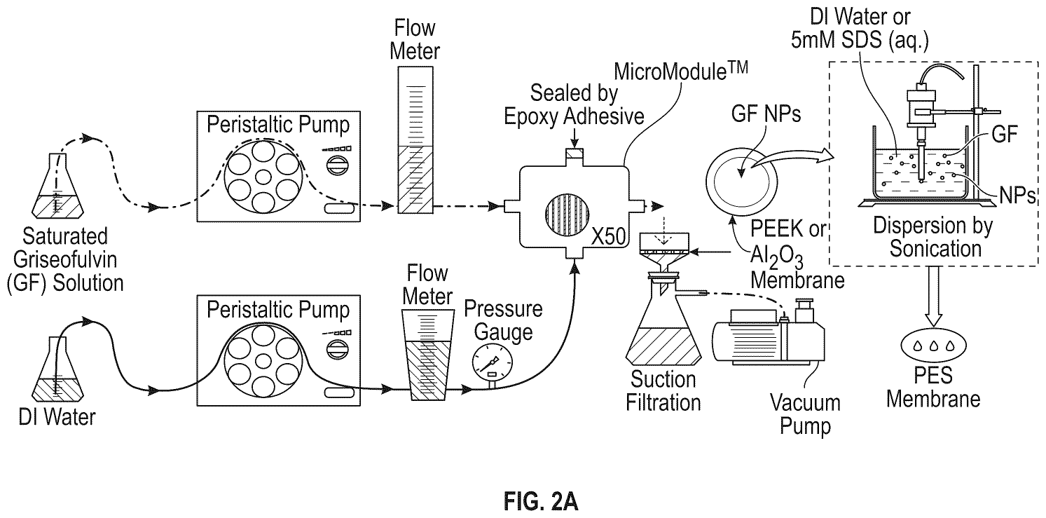

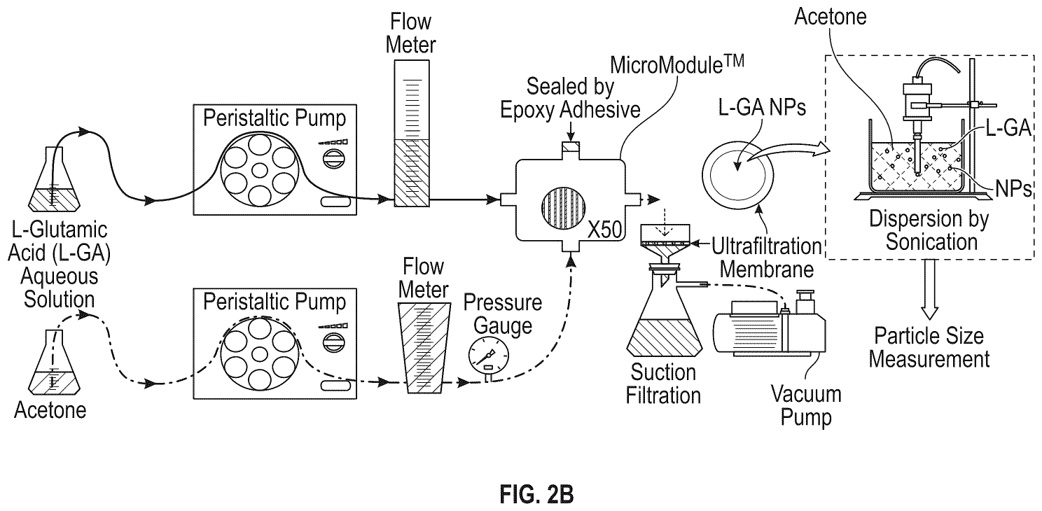

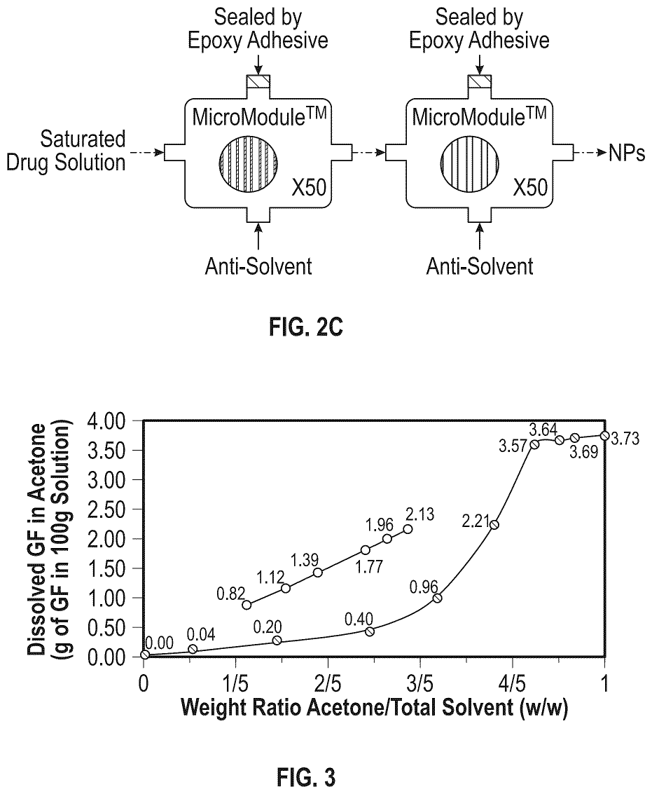

In the setup of FIG. 2A, pores of the hydrophobic polypropylene (PP) HFMs were wetted by first passing pure ethanol through the tube side using a peristaltic pump for a few minutes. This solution immediately wetted the membrane pores. Alternatively, for example, a 40% ethanol solution may be used for wetting purposes. This solution was slowly replaced by ethanol-water solutions of increasing water concentration and finally by pure water.

Then, pure water was kept flowing from the tube side using a peristaltic pump for a few hours until the HFM pores were thoroughly filled with water, at which time the pressure in the gauge would no longer drop and remained stable. This allowed anti-solvent water to flow through pores of the PP HFMs without applying a high pressure. The solution was taken out through one of the shell-side ends of the HFM module. The drug solution was then passed through one of the shell-side openings by a peristaltic pump. The resulting nanocrystal suspension was taken out of the HFM module through the other opening.

This solution introduction sequence is not needed for hydrophilic HFMs, such as nylon (See, Chen, D., et al., Continuous synthesis of polymer-coated drug particles by a porous hollow fiber membrane-based antisolvent crystallization, Langmuir 31, 432-441 (2015)) which are readily wetted by the anti-solvent water. In such implementations, the process can be initiated with anti-solvent flow through membrane pores and then immediately thereafter the feed solution may be introduced from the shell-side inlet. The suspension of crystals was taken out through the other shell-side opening. Once the membrane pores are wetted, they may be used in further operations without rewetting unless the module dries out in the interim.

In crystallization experiments with incoming saturated solutions, GF crystals precipitated continuously in the flowing shell-side solution and were collected at the outlet on the 0.02 μm PEEK/Al₂O₃ membranes used in the vacuum filtration system set up (Glass Vacuum Filter Holder, 47 mm, Model No. XX1514700, MilliporeSigma™, Billerica, MA). The crystal size distribution of GF was measured by resuspension of the dried samples of GF crystals collected from the 0.02 μm PEEK/Al₂O₃ membranes in DI water or 5 mM SDS aqueous solution.

Such crystals were collected, dried and weighed to estimate the yield in crystallization.

Additional crystallization experiments involved the following. During the experiments mentioned above, the solution going through the filter would often have a significant amount of drug in solution. Crystallization from such solutions was studied via the following procedure. Anti-solvent (water) was added to a saturated GF acetone solution to obtain a solution equivalent to that in the filtrate. After crystallization took place over 16 h+, the supernatant was filtered through a 0.45 μm Nylon filter to remove residual crystals acting as seeds. The filtered supernatant was used as the feed drug (GF) solution for experiments; deionized (DI) water was used as the anti-solvent.

After each GF crystallization experiment lasting for 1 h+, the module was cleaned by the following steps: 1) pure acetone at 25 mL/min flow rate was passed through the shell side for 2 min; 2) pure acetone at a flow rate of 25 mL/min was kept passing through the shell side while water was passed from the tube side to the shell side at 1 ml/min flow rate for 1 min; 3) then, pure acetone at a lower flow rate of 5 mL/min was passed through the shell side while water was passed from the tube side to the shell side at the flow rate of 1 mL/min for 10 min; 4) next, pure acetone at a flow rate of 25 mL/min was passed through the shell side while water was passed from the tube side to the shell side at a flow rate of 1 mL/min for 2 min; 5) lastly, only water was passed from the tube side to shell side at 4 mL/min for 20-30 min. Experiments were carried out continuously for 3 h to determine performance variability.

The crystal size distribution was determined as follows. The GF crystals collected on the membrane filters of 0.02 μm pore size were resuspended in a solution in a separate sonicated vessel; the solution was DI water or DI water containing 5 mM SDS. A Zetasizer Nano ZS Instrument (Malvern Panalytical Ltd., UK) was employed to analyze the particle size distribution (PSD) of the collected drug crystals in samples from this vessel using dynamic light scattering yielding the average crystal size D_{avg}. Crystal size was also determined from the drug suspension in the sonicated vessel into which the exiting drug suspension from the crystallizer was directly introduced bypassing the filtration process. The drug crystal containing suspension exiting the crystallizer was directly discharged into a sonicated reservoir containing DI water with or without 5 mM SDS solution. The crystals were collected by a flat polyethersulfone (PES) membrane filter.

Based on the mass and therefore the volume of the crystals for a given population density function, n(Dₚ), where the characteristic crystal diameter is Dₚ, the mean crystal size, D_{avg}, is defined as:

$${\overline{D}}_{{p}_{4 , 3}} = \frac{{\int }_{0}^{∞} {D}_{p}^{4} n ( {D}_{p} ) {dD}_{p}}{{\int }_{0}^{∞} {D}_{p}^{3} n ( {D}_{p} ) {dD}_{p}} = {D}_{avg} \qquad ( 1 )$$

Powder X-ray diffractograms were collected using a Bruker D8 Advance diffractometer equipped with a twin-twin optic and EIGER2 R 500K X-ray detector in reflection mode. The samples were scanned at ambient temperature in continuous mode from 3-40° 2θ with step size of 0.02° 2θ at 40 kV and 40 mA with CuKα radiation (1.54 Å). The incident beam path was equipped with primary soller slit 2.5-degree, secondary soller slit 4.0 and divergence slit 0.6 mm, in a fixed slit mode. Samples were prepared on a low background sample holder and placed on a spinning stage with a rotation time of 10 rev/min. Data were collected using DIFFRAC.MEASUREMENT CENTER (v. 7.5) and processed with DIFFRAC.EVA (v. 5.2). A Raman microscope (DXR, Thermo Scientific, Waltham, MA) was used to measure the molecular structure of GF crystals: the laser power was set at 10 mw; the laser wavelength was 780 nm.

For aqueous L-glutamic acid (L-GA) feed solution, antisolvent acetone readily wets PP HFMs, thereby causing the HFM to function in the same manner as an hydrophilic HFM. FIG. 2B provides an exemplary setup for processing of L-GA as the API. The principal differences relative to the exemplary setup for GF crystallization shown in FIG. 2A are as follows:
The API, L-GA, is dissolved in water;
A saturated aqueous solution is used as the feed solution.
Pure acetone is the anti-solvent coming in through the PP hollow fibers.
Acetone wets the membrane pores spontaneously; therefore, the extensive pore wetting steps used with GF crystallization are not needed.
L-GA crystals are resuspended in an acetone solution under sonication.

After each L-GA experiment lasting for about 1 h+, the HFM module was cleaned with DI water at a flow rate of 25 ml/min; the DI water was passed through the shell side for 2 min. Then, the flow rate of DI water was reduced to 5 ml/min; this was continued for 60 min.

The crystals of L-GA collected on an Al₂O₃ membrane filter of 0.02 μm pore size were resuspended in pure acetone in a separate sonicated vessel. A Zetasizer Nano ZS, Instrument (Malvern Panalytical Ltd., UK) was employed to analyze the particle size distribution (PSD) of the collected drug crystals in samples from this vessel using dynamic light scattering yielding the average crystal size D_{avg}.

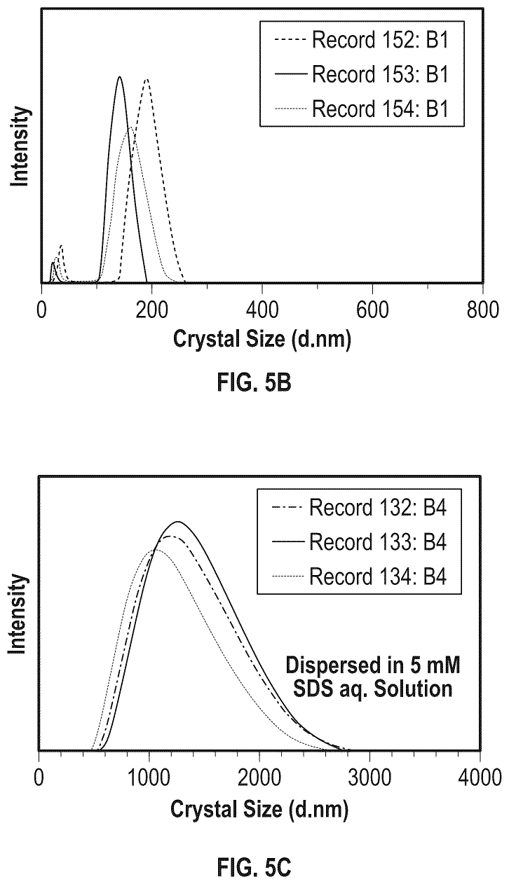

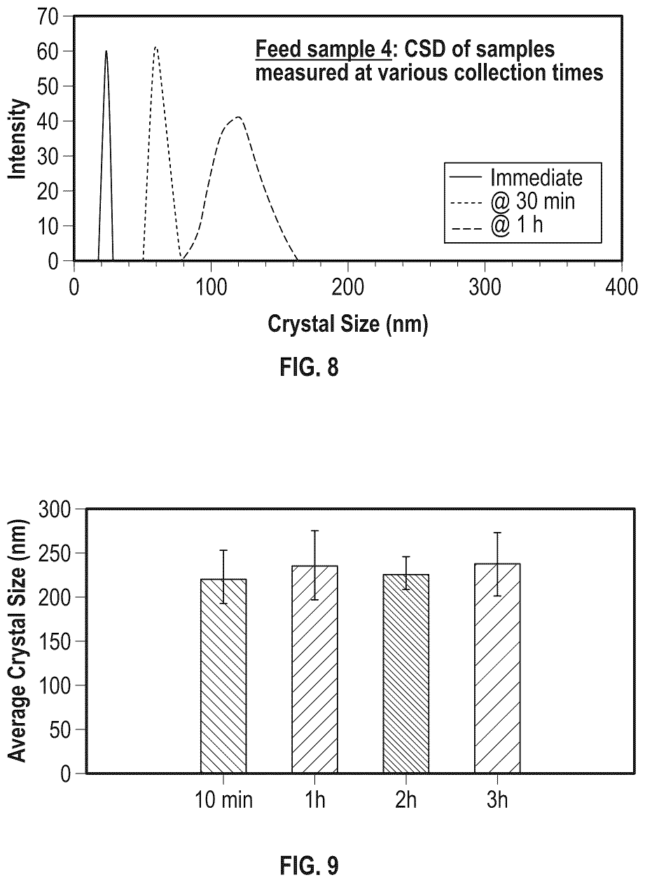

From the intensity distribution (in %) against the nm size of the crystals obtained from the Zetasizer Nano ZS instrument, a cumulative % intensity distribution (y) was developed against the nm size (x). This yielded a sigmoidal curve. This curve was fitted with the Gompertz model by the following equation (2):

$$y = a + ( b - a ) * exp ( - exp ( - c * ( x - d ) ) ) \qquad ( 2 )$$

where a, b, c, d are the parameters. The set of parameters yielding the highest R value was selected. From FIGS. 5A and 5C (discussed below), particular curves were selected and fitted using equation (2) to determine the parameters. Similarly, three curves in FIG. 8 (discussed below) were fitted using a 4-parameter logistic model described by the following equation (3):

$$y = d + ( a - d ) / ( 1 + {( x / c )}^{⋀} b ) \qquad ( 3 )$$

The results allow calculation of D₁₀, D₅₀, D₉₀ values of each distribution. The results are shown in Table 1 below.

TABLE 1
Estimation of Particular sizes for various % volume distributions of CSD

| | Equation (y is cumulative number %, | D₅₀ | D₁₀ | D₉₀ | Avg size, |
|---|---|---|---|---|---|
| Set (FIG.) | x is crystals size in nm) | nm | nm | nm | nm |
| 5a | y = 0.0054 + (102.1382 − 0.0054) *exp | 58 | 50 | 69 | 63 |
| | (−exp (−0.1493*(x − 55.4604))) | | | | |
| 5c | y = −0.0112 + (99.602 + 0.0112) *exp | 765 | 588 | 1047 | 872 |
| | (−exp (−0.0068*(x − 710.6950))) | | | | |
| 8- Immediate | y = 100.0346 + (−0.0769 − 100.0346)/ | 21 | 18 | 24 | 22 |
| | (1 + (x/20.6237) \hat{ }13.8663) | | | | |
| 8- 30 min | y = 99.99 + (−0.0646 − 99.99)/ | 56 | 48 | 66 | 61 |
| | (1 + (x/56.3982) \hat{ }14.2146) | | | | |
| 8- 1 h | y = 100.0052 + (−0.0695 − 100.0052)/ | 100 | 81 | 123 | 109 |
| | (1 + (x/99.9552)\hat{ }10.6087) | | | | |

FIG. 2C illustrates an experimental configuration of two Micromodule membranes used in series to increase crystallization yield. In this exemplary setup, each module was independently supplied by a separate pump delivering the required flow rate of the anti-solvent. One end of each module through which the HFMs are potted was sealed by epoxy. The suspension exiting the first module was fed into the second module which consumed a significant fraction of the residual dissolved solute via primarily crystal growth as well as nuclei formation.

The residence time (tᵣₑₛ) of the drug solution/suspension in the crystallizer was estimated as follows. Since residence times of the fluid elements being introduced into the flowing suspension by different HFMs at different axial locations of the HFM module (in the direction of mean shell-side flow) were different, an averaging procedure was used. An arithmetic average of two residence times was used corresponding to the incoming drug solution flow rate and the exiting suspension flow rate. The procedure for estimating the relevant shell-side volume is described below. Of note, the exiting suspension goes out through an arm of the cell adding to the residence time.

The weight of the cell under dry conditions was found to be We. The cell was then immersed into DI water in a beaker; using a pipette, DI water was pushed into the cell and micro-bubbles were blown out. A metal clip was attached to the top of the cell; this allowed the cell to sink to the bottom of the glass beaker where it was kept overnight. Then, using a pipette, DI water flushing was repeated to make sure that there were no microbubbles in the cell. Next, all four tail ends of the cell were sealed with a parafilm; the cell weight was determined as W_{f}. Then, using a pipette, DI water was removed from one tail of the cell and then this end was sealed with a parafilm. The cell was weighed again as Wₜ. Therefore, the weight of water in one tail of the cell is (W_{f}−Wₜ). The weight of the water in the cell without one tail is [W_{f}−W_{c}−(W_{f}−Wₜ)].

The solubility of GF in various acetone-water solutions at 25° C. is shown in FIG. 3. For any combination of flow rates of water and acetone, FIG. 3 is useful to quantify the supersaturation level potentially developed at the inlet locations of the membrane module in FIG. 2A for single-point mixing. Thus, it can guide selection of the two flow rates as one deliberates on the crystallization performance.

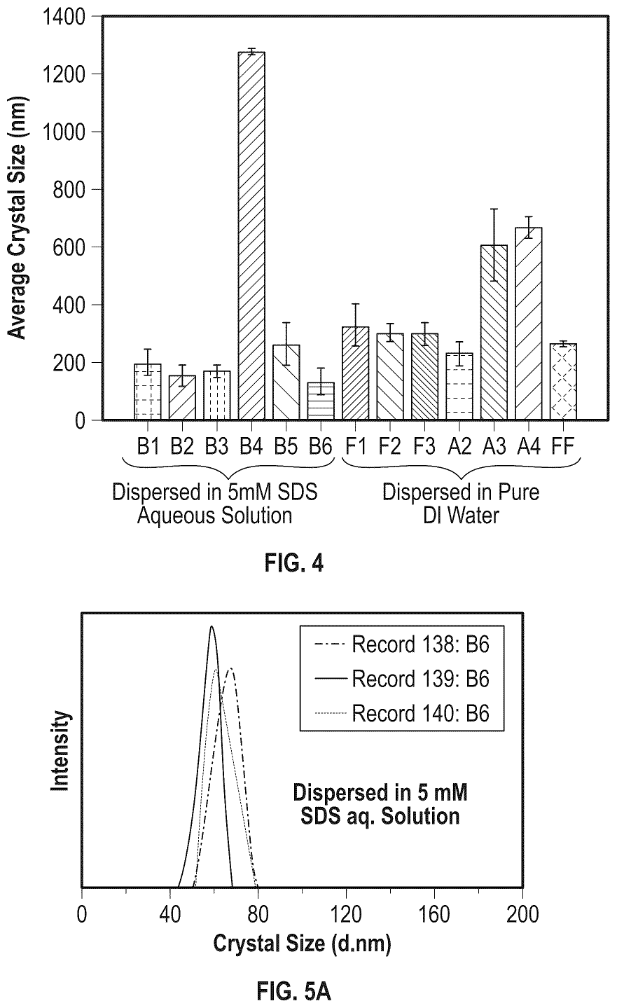

FIG. 4 illustrates the average GF crystal size and the standard deviation (SD) in the average size of the crystals collected from the filter at the exit of the module in FIG. 2A for a variety of flow rate combinations of the two streams and resuspended in a sonicated vessel for two types of aqueous solutions (DI water and 5 mM SDS solution). The variation from the average in duplicate runs is indicated by a so-called error bar at the top of each bar in FIG. 4. The values of each of the two liquid stream flow rates entering the module are shown in Table 2.

TABLE 2
Illustrative two liquid phase flow rates and
phase flow rate ratios for GF crystallization

| | | | Mass flow |
|---|---|---|---|
| Trial | GF acetone | Aqueous phase | rate ratio |
| No. | g/min | g/min | (acetone/water) |
| B1 | 4.28 | 3.75 | 1.1 |
| B2 | 8.74 | 3.75 | 2.3 |
| B3 | 13.55 | 3.75 | 3.6 |
| B4 | 3.65 | 7.37 | 0.5 |
| B5 | 6.66 | 7.37 | 0.9 |
| B6 | 9.61 | 7.37 | 1.3 |
| F1 | 4.31 | 3.26 | 1.3 |
| F2 | 5.29 | 3.26 | 1.6 |
| F3 | 8.24 | 3.26 | 2.5 |
| A2 | 6.14 | 6.26 | 1.0 |
| A3 | 6.14 | 10.55 | 0.6 |
| A4 | 6.14 | 11.77 | 0.5 |
| FF | 9.61 | 7.37 | 1.3 |

Two parameters are needed to understand the observed behavior: (1) the magnitude of each phase flow rate; (2) the flow rate ratio (FRR) of acetone phase to anti-solvent water phase. In general, a value higher than 1 of the FRR yields a lower average crystal size for the reported data. Further, a value smaller than 1 results in larger size crystals for the reported data. However, this result may be affected by the magnitude of each phase flow rate which affects the residence time, tᵣₑₛ. The device volume used to estimate tᵣₑₛ was 1.8 cm³ plus the volume of the connecting outlet for the suspension, 0.2 cm³. The volume of the connection bringing in the incoming drug solution did not have an impact on the result.

FIGS. 5A, 5B and 5C illustrate the CSD in each case from samples in runs B6, B1 and B4, respectively (FIG. 4). FIG. 5A shows the lowest average crystal size of ˜61 nm for a low tᵣₑₛ of 8.5 s. FIG. 5B shows an average crystal size of ˜198 nm for a somewhat higher tᵣₑₛ of 18.7 s. Here the mass FRRs (>1) are 1.3 and 1.14, respectively, reflecting a modest level of supersaturation. FIG. 5C shows a much larger average crystal size ˜1282 nm for a tᵣₑₛ value of 18.6 s, which was based on a mass FRR of 0.5, which introduced a much higher level of supersaturation. Characteristics of the CSD of a sample from some of the FIGS. 5A and 5C are shown in Table 1 above.

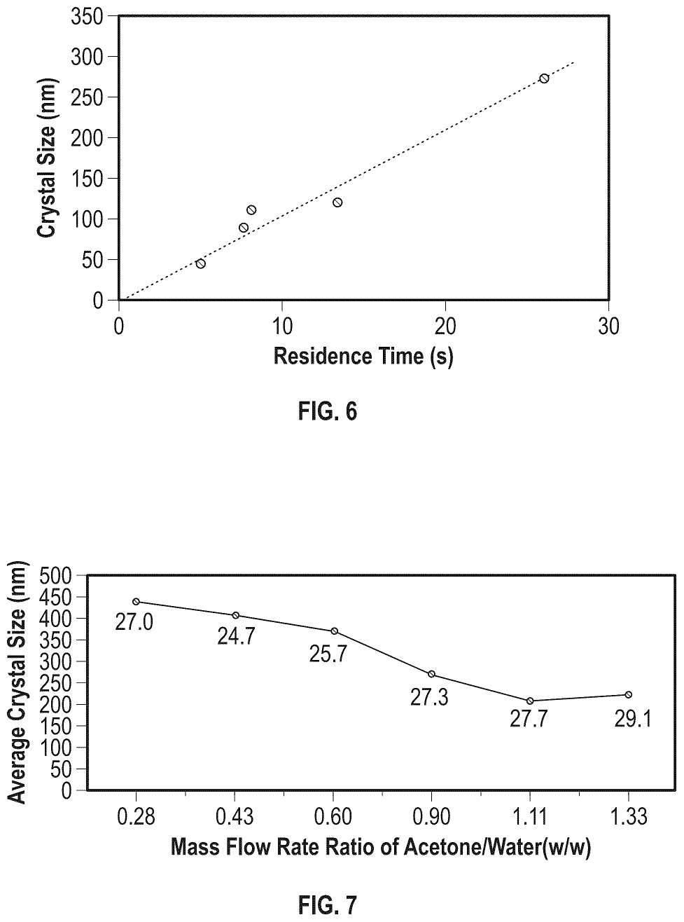

To develop a better understanding of these results, reference is made to FIG. 6. Here the supersaturation level is fixed in a series of experiments by having a mass FRR of 1.34±0.1 of feed acetone over anti-solvent water phase and then tᵣₑₛ is varied over a wide range at 25° C. by varying the total flow rate. The feed acetone solution was saturated in GF (=3.73 g GF/100 g acetone). It is clear that as tᵣₑₛ increases from a low value of ˜5 s to a high of ˜28 s, the average crystal size increases from ˜48 nm to 280 nm due to crystal growth. Considerable evidence shows: average crystal size increases as residence time increases. This result clearly demonstrates a facile technique for continuous nanocrystal production in a given nm size range. This could be used in at least two ways. For example, in CM of an API (implemented in small scale), a small number (e.g., 1 to 3) HFM modules operated in parallel can provide sufficient flow rate and crystal production rate. In other crystallization operations, this module can be used as a seed generator for larger scale batch/continuous crystallizers.

FIG. 7 illustrates the average crystal size obtained as a function of the mass flow rate ratio over a very small range of residence times showing the strong influence of supersaturation on the average crystal size. The residence time is influenced largely by the magnitude of the individual phase flow rates and their sum total. The final GF concentration as a result of mixing of the two streams in the anti-solvent crystallizer is shown by the noted straight line in FIG. 3 for each condition.

It shows that for each flow/mixing condition studied in FIG. 7, the operation takes place under significant supersaturation. Further, the lowest GF concentration developed by anti-solvent addition resulted in the largest crystal sizes.

Of note, the literature on anti-solvent crystallization involved final crystal dimensions of 3.5 to 63.5 μm [M. Stahl, et al., Reaction crystallization kinetics of benzoic acid, AIChE J., 47 (2001) 1544-1560; T. Rivera, et al., Continuous plug flow crystallization of pharmaceutical compounds, Ind. Eng. Chem. Process Des. Dev., 17 (1978) 182-188]. These studies show that as the feed solution to the anti-solvent ratio decreased at the mixing location, the average crystal size decreased. The systems and methods of the present disclosure show a different result, namely, the average crystal size increased, potentially based on the device design having a highly distributed feed to anti-solvent contact and other experimental features.

The growth of nanocrystals next to each HFM takes place over a distributed residence time because of the cross-flow device design; the development of the high supersaturation takes place also in a distributed fashion. The API-containing feed solution contacting the first layer of HFMs encounters a certain amount of anti-solvent coming in and developing a certain supersaturation level leading to nuclei formation. These nuclei will grow downstream to crystals of a certain size. When this suspension hits the second and the third layers of HFMs and so on further downstream, the supersaturation developed could be a bit higher or lower than that in the first layer depending on the nature of the solubility diagram and the anti-solvent introduction rate. However, the nuclei/crystals generated during contacting in the first and succeeding layers of HFMs will continue to grow. This process will go on further downstream with generally decreasing rates of nucleation due to consumption of the local supersaturation by nuclei development, crystal growth, and decreasing API concentration. However, for the lowest mass flow rate ratio of 0.28, there is much more supersaturation available along the whole device length in the mean flow direction and especially at the inlet location and therefore an opportunity for crystals to grow. As the crystallized suspension leaves the HFM contacting zone, there is also a device arm of small length through the bore of which the crystallizing solution flows; such a flow adds to the residence time, thereby increasing crystal growth. Further, crystal samples grow during rapid collection from the filter via consumption of residual supersaturation.

In the experimental results using one HFM-based AsCr module, the GF crystal yield % based on the input rate is generally less than 50%. Therefore, a considerable fraction of GF did not crystallize and remained in the solution passing through the filter collecting already-formed crystals; however, its concentration would be reduced due to any growth of those crystals from contact with the GF solution during filtration. As described above, a method is provided for preparing saturated GF solutions in acetone-water mixtures with GF concentrations lower than that of saturated GF solution in acetone only. Crystallization results from such saturated GF solutions in acetone-water mixtures similar to those passing through the filter are discussed now.

Table 3 lists the numerical values of the yield of GF nanocrystals from saturated feed solutions in acetone-water mixtures having lower GF concentrations under different mass flow rate ratios of acetone to water (one example is shown in FIG. 8). In trials No. 1 and No. 2, the feed solutions were saturated with GF in an acetone-water mixture (68.7% w/w of acetone). The yield in trial No. 2 surpassed that of trial No. 1 because the antisolvent percentage in Trial No. 2 exceeded that of Trial No. 1. In Trial No. 3, there was insufficient collection of GF crystals due to the low ratio of antisolvent (water) to acetone introduced into the system. Trial No. 4 resulted in the collection of very fine crystals due to a decrease in the FRR, by maintaining the same antisolvent phase mass flow rate and reducing the saturated GF feed solution flow rate in an acetone-water mixture (50% w/w of acetone). The filtration time to collect these fine GF crystals from the suspension containing small amounts of GF crystals was significantly extended.

TABLE 3
Yield of GF nanocrystals from lower
API concentration feed solution

| | Saturated GF | | Anti-solvent | Mass flow | |
|---|---|---|---|---|---|
| | solution — | GF | phase | rate ratio | |
| Trial | acetone weight | solution, | (water), | (GF solution/ | |
| No. | ratio in feed | g/min | g/min | water) | Yield |
| 1 | *68.7% | 14.7 | 6.1 | 2.4/1 | 47% |
| 2 | 68.7% | 9.5 | 9.5 | 1/1 | 55.2% |
| 3 | 50% | 8.7 | 9.5 | 1/1.1 | N/A |
| 4 | 50% | 1.0 | 9.5 | 1/9.5 | 15% |

*68.7% (wt/wt) acetone in a mixture of acetone and water

FIG. 8 illustrates the antisolvent crystallization performance of such simulated filtrates (shown in Table 3) containing a much lower level of solute as the feed into the crystallization device. Samples were collected from the filtration cup at different periods of time from the start. Since nanocrystals of very small size were being formed and a 0.02 μm Al₂O₃ membrane was used in suction filtration, filtration rates were slow: the solution volume in the filtration cup slowly increased. Samples collected from the filtration cup at three (3) different times were analyzed for CSD.

FIG. 8 shows that as the filtration began, crystals of average size ˜25 nm were being produced. Then, as the solution volume increased slowly in the filtration cup, the average crystal size grew to ˜55 nm and ˜110 nm at 30 min and 1 h, respectively. Thus, by reducing the API concentration in the acetone-water feed, it is feasible to produce very small size GF nanocrystals. FIG. 9 shows steady performance of the disclosed membrane-based anti-solvent crystallization. An experiment was conducted without interruption over 3 hours at a particular mass flow rate ratio (˜1.33) to determine whether product nanocrystal size was affected by the extended time period of the run. The average crystal size was around 230 nm with very little variation with time. Thus, continuous steady-state crystallization has been demonstrated. Details of this run and the numerical values of the nanocrystals are provided in Table 4.

TABLE 4
Average crystal size of GF obtained at different times during the 3-hour long run

| | | | Total | Mass | | | Total | Vol. | | | |
|---|---|---|---|---|---|---|---|---|---|---|---|
| | | | mass | flow | | | Vol. | flow | Avg. | | |
| | | | flow | rate | | | flow | rate | crystal | SD*** | |
| | | | rate | ratio | | | rate | ratio | size | from | Avg. |
| Time | $GF Ac * \frac{g}{min}$ | $Aq . phase \frac{g}{min}$ | $\frac{g}{min}$ | $\frac{A c}{A q * *}$ | $GF Ac . \frac{mL}{min}$ | $Aq . phase \frac{mL}{min}$ | $\frac{mL}{min}$ | $\frac{A c}{A q}$ | D₅₀ nm | avg. nm | tᵣₑₛ s |
| 10 | 2.68 | 2.02 | 4.70 | 1.33 | 3.35 | 2.02 | 5.37 | 1.66 | 223 | 55 | 29.1 |
| min | | | | | | | | | | | |
| 1 h | | | | | | | | | 235 | 79 | |
| 2 h | | | | | | | | | 226 | 38 | |
| 3 h | | | | | | | | | 238 | 71 | |

*Ac-acetone;
**Aq-water;
***SD-Standard deviation

To increase the crystallization yield, experiments were performed using the configuration of two modules in series shown in FIG. 2C. The results are presented in Table 5. For Trial #1, the fractional yield varied between 0.74 and 0.89. For Trial #3, the fractional yield was a very high value, 0.98. This is a significant result. However, it is noted that the tightly packed HFMs caused a significant increase in pressure for the data shown in the second stage, especially when the flow rate of GF acetone solution increased. The packing fraction of the HFMs in this HFM module is ˜0.43 which is on the high side. Since most of the GF could be precipitated out with two stages in series, the precipitated nanocrystals would start clogging the shell-side of the second module. The clogging caused an increase in pressure and also affected subsequent crystal precipitation. The issue is addressable by utilizing a loosely packed hollow fiber membrane module design for the second stage.

TABLE 5
Yield of GF nanocrystals when two modules are in series

| | | | Flow | | | | Overall | | | Dispersed | | | |
|---|---|---|---|---|---|---|---|---|---|---|---|---|---|
| | | Aqueous | rate | Resi- | Aqueous | Total | flow rate | | | in DI water | Average | SD | |
| | GF | phase - | ratio | dence | phase - | flow | ratio | Residence | | or 5 | particle | from | |
| Trial | acetone, | I, | (acetone/ | times | II, | rate | (acetone/ | times | Anti- | mM SDS | size | avg. | |
| No. | mL/min. | mL/min | water) | 1^{st} stage | mL/min | mL/min | water) | 2^{nd} stage | solvent | aqueous sol. | D₅₀, nm | nm | Yield |
| Trial | 3.25 | 1.93 | 1.68 | 30.0 | 6.80 | 11.98 | 0.372 | 23.5 | DI water | 5 mM SDS | 344 | 13 | 74- |
| #1 | | | | | | | | | | (aq.) | | | 89% |
| Trial | 3.96 | 1.82 | 2.17 | 25.5 | 8.99 | 14.78 | 0.366 | 19.2 | DI water | 5 mM SDS | 685 | 50 | N/A |
| #2 | | | | | | | | | | (aq.) | | | |
| Trial | 2.89 | 1.93 | 1.35 | 33.3 | 6.92 | 11.71 | 0.327 | 25.9 | DI water | 5 mM SDS | 194 | 68 | 98% |
| #3 | | | | | | | | | | (aq.) | | | |

It is of interest to assess the level of process intensification being achieved in this device. Exact quantification is difficult because crystallization rates are dependent on systems under consideration. However, there is a certain amount of information about the performance of the GF system. Looking at Tables 3 and 5, one can assume ˜50% yield from feed solution flow rates between ˜5 to 10 cm³/min. Further, the internal volume of the crystallizer occupied by the liquid phase is ˜2 cm³. Therefore, the fractional yield/volume is ˜(0.5/2 cm³), i.e., 0.25/cm³ for feed flow rates of 5-10 cm³/min. This value appears to be quite high.

The following information is available for a Continuous Oscillatory Baffled Crystallizer (COBC) [See, Brown, C. J., et al., Characterization and modelling of antisolvent crystallization of salicylic acid in a continuous oscillatory baffled crystallizer, Chemical Engineering and Processing, 97 (2015) 180-186]. For a feed solution flow rate of 40 cm³/min and antisolvent flow rate varying between 40-80 cm³/min, assume a yield of 0.50. The dimensions of the feed solution baffled tube are: 15 mm outer diameter, 700 mm length; there is a similar baffled tube for the antisolvent and then a similar tube for the downstream crystallizer. Assume the internal diameter of each tube is 10 mm; then the internal volume of the solution in any one leg of the crystallizer is 54.5 cm³. Therefore, the yield per unit volume is (0.5/163.5 cm³)=0.003/cm³ for a feed flow rate 4-8 times larger. If one increases feed flow rate 8 times to 40 cm³/min for this invention, the volume required is 16 cm³. Thus, yield per/cm³ of HFM crystallizer (0.5/16 cm³=0.031) is ˜10 larger than that of the COBC; if the yield in [C. J. Brown et al., 2015] is assumed to be 1, the HFM device value is a factor 5 larger. It is not known that this performance can be achieved by a larger HFM device; however, this level of performance can be achieved using a few small HFM devices in parallel.

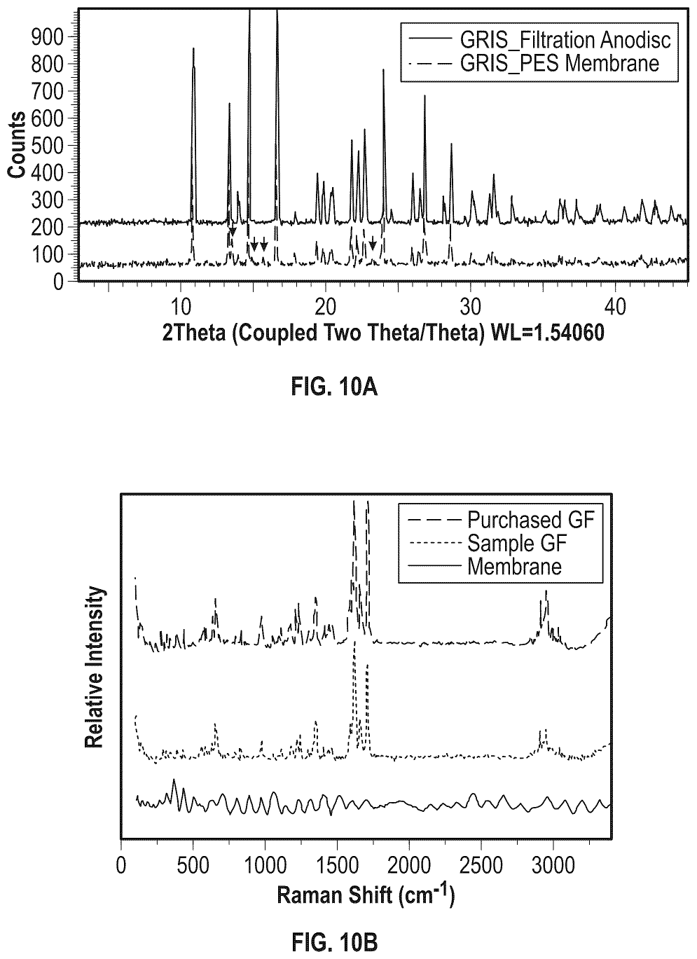

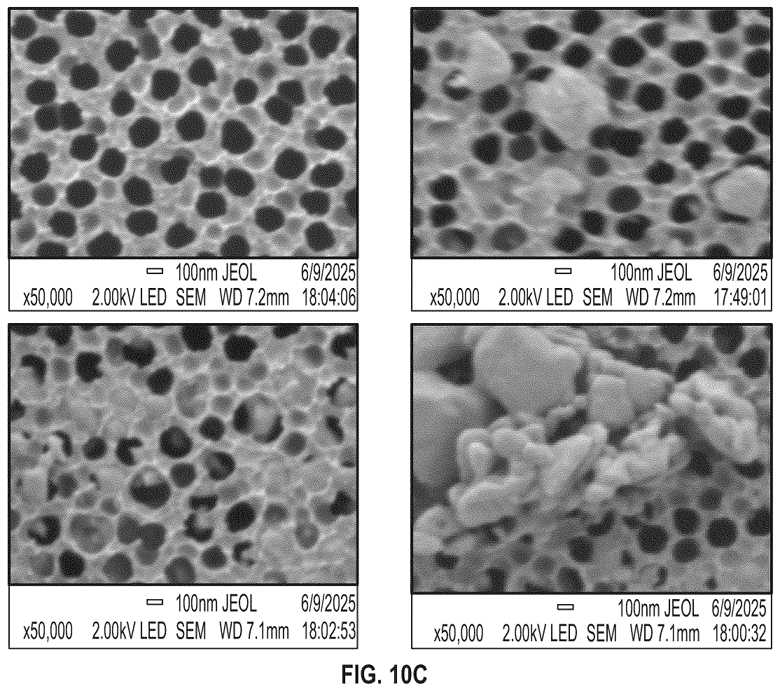

Two PXRD patterns of GF crystals shown in FIG. 10A are essentially identical to results obtained previously [See, D. Chen, et al., Continuous synthesis of polymer-coated drug particles by a porous hollow fiber membrane-based antisolvent crystallization, Langmuir, 31 (2015) 432-441]. However, those collected on the PES filtration membrane have four (4) additional peaks. These correspond to GF form IV among five polymorphs of griseofulvin in the Cambridge Data Base [See, C. R. Groom, et al., The Cambridge Structural Database, Acta Cryst., B72 (2016) 171-179.]. It is possible that during the nanocrystal formation (in the PES membrane experiment), the metastable form may have been isolated and in the due course of time before the samples were analyzed by PXRD, it may have already been transformed into the most stable form (Form 1). The Raman spectra shown in FIG. 10B are also identical to results obtained earlier [See, Chen et al. (2015)].

FIG. 10C provides SEM images for GF crystallization filter and GF crystals on top of it: Top left-alumina filter; top right-GF nanocrystals on filter; bottom left-smaller crystals going through filter pores; bottom right-large collection of GF crystals on the alumina filter. Specifically, the SEM of an Al₂O₃ membrane of 0.02 μm pore size used to filter the suspension exiting the crystallizer is shown in top left of FIG. 10C. Next, in three more SEMs, we have shown GF nanocrystals collected on this filter; the dimensions and the shape of the GF nanocrystals are clearly visible. Some nanocrystals are going through the pores. While some nanocrystals appear jaggedly rounded, there are a few with tetragonal shape which is the shape of Form I of GF.

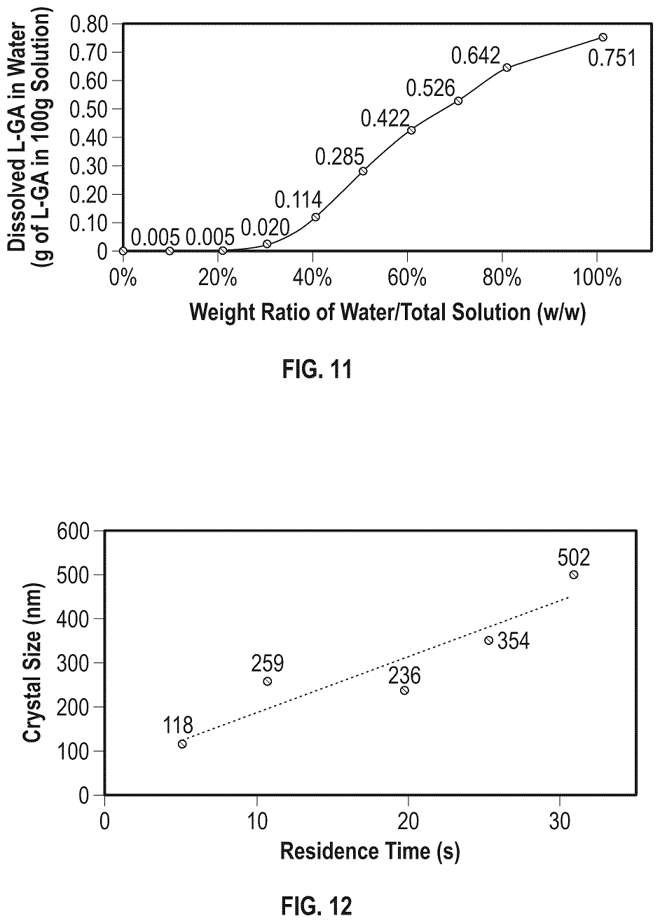

L-glutamic acid (L-GA) has a significant solubility in water while acetone is an anti-solvent. The solubility of L-GA in various water-acetone solutions at 25° C. is shown in FIG. 11. A saturated solution of L-GA in water (7.5 mg/mL) was passed through the shell side of the cross-flow module while pure acetone was passed through the bore of the HFMs. FIG. 12 illustrates the nanocrystal size obtained in a number of runs where the mass flow rate ratio of water phase flow rate to anti-solvent acetone phase flow rate was fixed at around 1.3 as the residence time was varied between 5.1 s and 31.5 s. As the residence time increased, the average nanocrystal size increased from 118 to 502 nm. This illustrates the expected effect of increased tᵣₑₛ on the crystal size. This behavior is quite similar to that demonstrated for GF crystallization (see, e.g., FIG. 6).

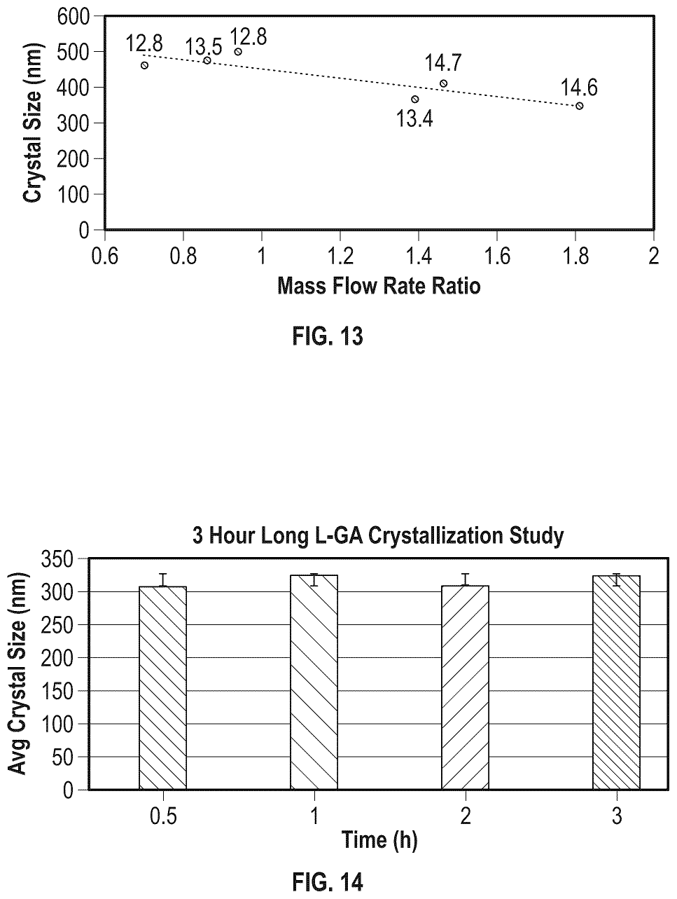

FIG. 13 illustrates results for cases where the residence time was fixed around 13 s and the mass flow rate ratio was varied between 0.7 and 1.81. As shown therein, an increase in the average L-GA crystal size is demonstrated with decreasing FRR, as also demonstrated for GF crystallization (see, e.g., FIG. 7). Therefore, generation of high supersaturation in a distributed fashion along the main flow direction allows crystal growth to overshadow high nucleation rate due to high supersaturation.

In like manner to the experimental work shown in FIG. 9 for griseofulvin, an extended term study of the continuous crystallization process was also undertaken for L-GA. The results are shown in FIG. 14. The data show that over a period of 3 hours, the average crystal size remained essentially unchanged at around 317 nm. This result demonstrates that the process is stable over time.

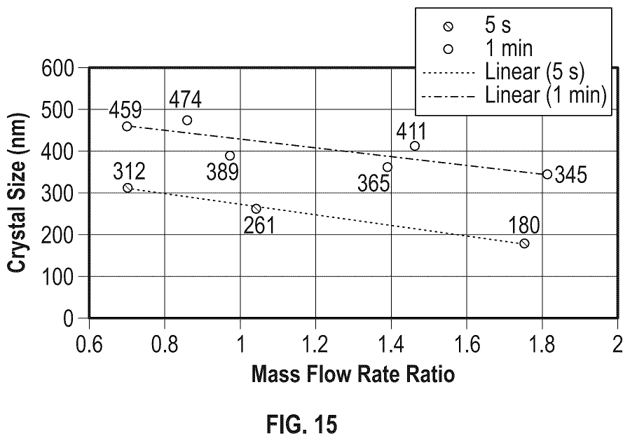

Of note, in all experiments conducted to produce nanocrystals, the suspension leaving the HFM module through the exiting tail of the module was discharged onto a microporous membrane in a vacuum filter holder. The suspension on top of this flat filtration membrane in the filter holder allows crystals in the suspension to grow since the filtration rate was not very high. In most experiments, the suspension was discharged onto the filtration membrane for about 1 min, at which time no more suspension was discharged into it and the filtration assembly was opened to take the membrane out with all the crystals on top of it. In other experiments, the suspension was discharged into the filtration assembly only for 5 s to cut down on the crystal growth in the filtration cup. FIG. 15 illustrates data for this latter approach for L-GA for 5 s in the filtration cup vs. 1 min in the filtration cup. As is apparent, the crystal sizes are considerably smaller for the 5 s collection time. Further, due potentially to size-independent growth rate, the two lines are essentially parallel regardless of the flow rate ratio and corresponding supersaturation generated.

The data illustrated here were gathered using porous HFMs of polypropylene. Solvent-resistant hydrophilic HFMs of other polymers e.g., polyamide (nylon), regenerated cellulose, polyethersulfone, a block copolymer of a polyamide and a polyether (e.g., PEBAX), hydrophilized polyetheretherketone (PEEK) and polyacrylonitrile, can also be used. Solvent-resistant hydrophobic HFMs of polymers of polytetrafluoroethylene (PTFE), polyetheretherketone (PEEK), polyethylene, and polyvinylidene fluoride (PVDF), for example, may also be used.

Disclosed and demonstrated herein is the AsCr performance of a porous HFM module design where the bulk API-containing solution flowing on the shell side is in cross flow vis-à-vis the HFMs through the bore of which the anti-solvent is injected into the shell side. This design allows for a very short flow length of the API-containing solution achieving residence times as low as 5 s and small dimensions of the resulting nanocrystals. Micromixing is also very efficient in such a device due to the superposition of mixing by crossflow around HFMs on the mixing induced by injection of the antisolvent through membrane pores. These results have been demonstrated using the crystallization of GF from its solution in acetone with water as anti-solvent and L-GA from its solution in water with acetone as the anti-solvent. For a fixed flow rate ratio, nanocrystals grew with increased residence time. Due to the distributed nature of supersaturation development along the module length in the mean flow direction, decreasing flow rate ratio resulting in increased supersaturation, however, yielded increased nanocrystal size. Stable GF nanocrystal production performance has been demonstrated, e.g., via an experiment lasting 3 hours.

An important difference between the crossflow design of the present device and the parallel flow design of earlier HFM modules is the following. The cross flow of the shell-side liquid aided by the anti-solvent injection can and does remove any nanocrystal around any HFM to the main flow. This prevents their accumulation on the HFM surface and longer-term fouling development. In a parallel flow module, nanocrystals get pushed downstream only along the membrane surface and can still remain on the HFM surface if the antisolvent injection rates are not high. Therefore, the possibility of membrane fouling is considerably higher in parallel flow designs. None was observed here with a cross-flow design in the extended term experiments.

Employing two HFM modules in series, GF crystallization yields as high as 0.98 have been achieved while maintaining an average nanocrystal size of approximately 200 nm. A small compact HFM-based device can continuously produce nanocrystals of APIs in ˜40-500 nm+size range with a compelling yield.

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

What is claimed is:

1. A method for anti-solvent crystallization of material, comprising the steps of:
providing a hollow fiber membrane module including a plurality of porous hollow fibers arranged in a cross-flow configuration relative to a shell-side fluid flow;
flowing a solution containing the material through a shell-side of the hollow fiber membrane module; and
flowing an anti-solvent through a tube-side of the hollow fiber membrane module;
whereby the anti-solvent permeates through pores of the hollow fibers into the shell-side of the hollow fiber membrane module to crystallize the material from the solution.

2. The method of claim 1, wherein the anti-solvent crystallization is a continuous process.

3. The method of claim 1, wherein the material is an active pharmaceutical ingredient (API).

4. The method of claim 1, further comprising a second hollow fiber membrane module in series with the hollow fiber membrane module.

5. The method of claim 1, wherein the porous hollow fibers are fabricated from a fiber material selected from the group consisting of polypropylene, polyamide, regenerated cellulose, polyethersulfone, polyacrylonitrile, and a block copolymer of a polyamide and a polyether.

6. The method of claim 1, wherein the porous hollow fibers are fabricated from a solvent-resistant hydrophobic material.

7. The method of claim 6, wherein the solvent-resistant hydrophobic material is selected from the group consisting of polypropylene, polytetrafluoroethylene (PTFE), polyetheretherketone (PEEK), polyethylene, and polyvinylidene fluoride (PVDF).

8. The method of claim 1, wherein the porous hollow fibers are fabricated from a solvent-resistant hydrophilic material.

9. The method of claim 8, wherein the solvent-resistant hydrophilic material is selected from a group consisting of polyamide, regenerated cellulose, hydrophilized PEEK, polyacrylonitrile, and hydrophilized polyetherimide.

10. The method of claim 1, wherein the material crystallized from the solution defines a nanocrystal.

11. The method of claim 1, wherein the residence time of the solution containing the material is 1-180 seconds.

12. A hollow fiber membrane module for continuous anti-solvent crystallization, comprising:
a housing;
a plurality of porous hollow fibers arranged within the housing, wherein the hollow fibers are oriented substantially perpendicular to a shell-side flow direction;
at least one shell-side inlet in the housing sized to introduce a solution containing a material; at least one shell-side outlet in the housing sized to collect a crystal suspension;
at least one tube-side inlet sized to introduce an anti-solvent into bores of the hollow fibers,
wherein the hollow fibers are configured to allow permeation of the anti-solvent from the fiber bores into a shell-side of the module to crystallize the material from the solution.

13. The hollow fiber membrane module of claim 12, wherein the porous hollow fibers are fabricated from a fiber material selected from the group consisting of polypropylene, polyethylene, polyamide, regenerated cellulose, polyethersulfone, polyacrylonitrile, and a block copolymer of a polyamide and a polyether.

14. The hollow fiber membrane module of claim 12, wherein the porous hollow fibers are fabricated from a solvent-resistant hydrophobic material.

15. The hollow fiber membrane module of claim 14, wherein the solvent-resistant hydrophobic material is selected from the group consisting of polytetrafluoroethylene (PTFE), polyetheretherketone (PEEK), polyethylene, polypropylene and polyvinylidene fluoride (PVDF).

16. The hollow fiber membrane module of claim 12, wherein the porous hollow fibers are fabricated from a solvent-resistant hydrophilic material.

17. The hollow fiber membrane module of claim 16, wherein the solvent-resistant hydrophilic material is selected from the group consisting of polyamide, regenerated cellulose, hydrophilized PEEK, polyacrylonitrile, and hydrophilized polyetherimide.

18. The hollow fiber membrane module of claim 12, further comprising a second hollow fiber membrane module in series with the hollow fiber membrane module.
